(19) United States
(12) Patent Application Publication
(10) Pub. No.: US 2022/0037036 A1
(43) Pub. Date: Feb. 3, 2022

(54) METHODS AND SYSTEMS FOR MITIGATING TRANSMISSION OF A CONTAGIOUS INFECTION WITHIN A SHARED WORKSPACE

(71) Applicant: Léon DesRoches, Dieppe (CA)

(72) Inventor: Léon DesRoches, Dieppe (CA)

(21) Appl. No.: 17/388,981

(22) Filed: Jul. 29, 2021

Related U.S. Application Data
(60) Provisional application No. 63/059,351, filed on Jul. 31, 2020.

(51) Int. Cl. G16H 50/80; H04L 12/58

(52) U.S. Cl. CPC G16H 50/80; H04L 51/04

(57) ABSTRACT

Systems and methods are disclosed for contact tracing in a shared workspace. One of the methods include: receiving an indication that a user of a plurality of users has symptoms associated with a contagious infection; identifying exposed users of the plurality of users that were in a contagious proximity to the user during a preceding period and sending a notification to each of the exposed users to inform of contact with the user. Systems and methods are also disclosed for obtaining completion of a contagion control procedure, the methods being performed in connection with a workstation. One of the methods include: transmitting control signals that instruct one or more workstation actuators to move a workstation tabletop to an uncomfortable height; detecting presence of a user at the workstation; and in response to determining that the user has not completed the contagion control procedure, maintaining the tabletop in the uncomfortable height.

### CROSS-REFERENCE TO RELATED APPLICATION(S)

This application claims priority to U.S. Provisional Application No. 63/059,351 filed Jul. 31, 2020, the entire disclosure of which is hereby incorporated by reference in its entirety.

### FIELD

This application relates generally to mitigation of health concerns within a shared workspace. In one aspect, methods and systems relate to contact tracing of a contagious infection in a shared workspace having a plurality of workstations. In another aspect, methods and systems relate to obtaining completion of a contagion control procedure in connection with a power-actuated workstation.

### INTRODUCTION

Shared workstations, such as, for example, office buildings and classrooms, present challenges when it comes to mitigating transmission of contagious infections between occupants (e.g. co-workers or classmates). The close proximity of occupants that is common within these settings increase the risk of transmission.

Workstations, such as, for example, desks and tables, are commonplace in shared workplaces. Multiple workstations may be distributed within a shared workspace, e.g. one per cubicle, one per office, open plan, etc. In some cases, the workstations may be power-actuated workstations. A power-actuated workstation may include one or more actuators, such as electric motors or solenoids, that drive one or more work surfaces (e.g. tabletop or keyboard tray) to move (e.g. linearly, rotationally, or accurately) relative to one or more axes of the workstation. For example, a sit-stand desk may include one or more vertical columns that support a tabletop and that are power extensible to move the tabletop vertically between a seated height and a standing height.

### DRAWINGS

FIG. 1 is a bottom perspective view of an example power-actuated workstation having one workstation actuator;

FIG. 2A is a side view of another example power-actuated workstation with a tabletop in a raised and rearward position;

FIG. 2B is a side view of the power-actuated workstation of FIG. 2A, with the tabletop in a raised and forward position;

FIG. 2C is a side view of the power-actuated workstation of FIG. 2A, with the tabletop in a lowered and forward position;

FIG. 2D is a side view of the power-actuated workstation of FIG. 2A, with the tabletop in a lowered and forward position;

FIG. 3 is a front perspective view of another example power-actuated workstation having two workstation actuators;

FIG. 4 is a schematic illustration of a server computer communicatively coupled to a workstation controller, a user device, a biometric sensor, and a remote storage device;

FIG. 5 is a flowchart illustrating an example process executed by one or more processors for obtaining completion of a contagion control procedure;

FIGS. 6A to 6F depict a smartphone displaying successive steps of an example contagion control procedure on its screen;

FIG. 7 is a flowchart illustrating an example method of contact tracing in a shared workspace having a plurality of workstations;

FIG. 8 is front perspective view of an example workstation;

FIG. 9 is a side perspective view of an example visitor registration device;

FIG. 10 is a schematic illustration of a floor plan showing the location of workstations in a shared workspace.

### SUMMARY

In a first aspect, a method of obtaining completion of a contagion control procedure is disclosed. The method can be performed in connection with a workstation having a tabletop and one or more actuators operable to move the tabletop. The method includes: detecting presence of a user at the workstation, the user having a seated tabletop height and a standing tabletop height stored in association with the workstation; transmitting, to the one or more actuators, control signals that instruct the one or more actuators to move the tabletop to an uncomfortable tabletop height between the seated tabletop height and the standing tabletop height; and in response to determining that the user has not completed the contagion control procedure, inhibiting the tabletop from moving away from the uncomfortable tabletop height in accordance with at least one of manual user instructions and automated user preferences.

In another aspect, a method of obtaining completion of a contagion control procedure is disclosed. The method can be performed in connection with a workstation having one or more actuators operable to move a tabletop of the workstation. The method includes: transmitting, to the one or more actuators, control signals that instruct the one or more actuators to move the tabletop to an uncomfortable tabletop height between 85 cm and 105 cm above the floor; detecting presence of a user at the workstation; and in response to determining that the user has not completed the contagion control procedure, inhibiting the tabletop from moving away from the uncomfortable tabletop height in accordance with at least one of manual user instructions and automated user preferences.

In another aspect, a system is disclosed for obtaining completion of a contagion control procedure in connection with a workstation having a tabletop and one or more actuators operable to move of the tabletop. The system includes: a workstation controller configured to send control signals to the one or more actuators to move the tabletop; a server computer communicatively coupled to the workstation, each of the workstation controller and the server computer having one or more processors, the processors of the workstation controller and server computer configured to collectively: detect presence of a user at the workstation, the user having a seated tabletop height and a standing tabletop height stored in association with the workstation; transmit, to the one or more actuators, control signals that instruct the one or more actuators to move the tabletop to an uncomfortable tabletop height between the seated tabletop height and the standing tabletop height; and in response to determining that the user has not completed the contagion control procedure, inhibit the tabletop from moving away from the uncomfortable tabletop height in accordance with at least one of manual user instructions and automated user preferences.

In another aspect, a computer-readable medium is disclosed that includes instructions executable by one or more processors. The instructions when executed configure the one or more processors to collectively: detect presence of a user at a workstation, the user having a seated tabletop height and a standing tabletop height stored in association with the workstation; transmit, to one or more actuators of the workstation, control signals that instruct the one or more actuators to move a tabletop of the workstation to an uncomfortable tabletop height between the seated tabletop height and the standing tabletop height; in response to determining that the user has not completed a contagion control procedure, inhibit the tabletop from moving away from the uncomfortable tabletop height in accordance with at least one of manual user instructions and automated user preferences.

In another aspect, a system is disclosed for contact tracing in a shared workspace having a plurality of workstations. The system includes: a server computer having one or more processors; each of the plurality of workstations having a workstation controller communicatively coupled to the server computer, each workstation controller having one or more processors; the server computer and the workstation controllers of the plurality of workstations configured to collectively: (i) receive an indication that a symptomatic user of a plurality of users has one or more symptoms associated with a contagious infection; (ii) identify exposed users of the plurality of users that were in a predetermined contagious proximity to the symptomatic user during a predetermined preceding period by (a) identifying one or more contaminated workstations of the plurality of workstations where the symptomatic user was identified as present during the predetermined preceding period; (b) identifying each other workstation of the plurality of workstations, which are located in the predetermined contagious proximity to the one or more contaminated workstations, as exposed workstations; and (c) identifying all other users of the plurality of users, who were identified as present at any of the exposed workstations or the contaminated workstations during the predetermined preceding period, as the exposed users; and (iii) send a notification to each of the exposed users indicating they were in the predetermined contagious proximity to the symptomatic user.

In another aspect, a method of contact tracing in a shared workspace having a plurality of workstations is disclosed. The method includes: (i) receiving an indication that a symptomatic user of a plurality of users has one or more symptoms associated with a contagious infection; (ii) identifying exposed users of the plurality of users that were in a predetermined contagious proximity to the symptomatic user during a predetermined preceding period by (a) identifying one or more contaminated workstations of the plurality of workstations where the symptomatic user was identified as present during the predetermined preceding period; (b) identifying each other workstation of the plurality of workstations, which are located in the predetermined contagious proximity to the one or more contaminated workstations, as exposed workstations; and (c) identifying all other users of the plurality of users, who were identified as present at any of the exposed workstations or the contaminated workstations during the predetermined preceding period, as the exposed users; and (iii) sending a notification to each of the exposed users indicating they were in the predetermined contagious proximity to the symptomatic user.

In another aspect, a computer-readable medium is disclosed that includes instructions executable by one or more processors. The instructions when executed configure the one or more processors to collectively: (i) receive an indication that a symptomatic user of a plurality of users has one or more symptoms associated with a contagious infection; (ii) identify exposed users of the plurality of users that were in a predetermined contagious proximity to the symptomatic user during a predetermined preceding period by (a) identifying one or more contaminated workstations of a plurality of workstations where the symptomatic user was identified as present during the predetermined preceding period; (b) identifying each other workstation of the plurality of workstations, which are located in the predetermined contagious proximity to the one or more contaminated workstations, as exposed workstations; and (c) identifying all other users of the plurality of users, who were identified as present at any of the exposed workstations or the contaminated workstations during the predetermined preceding period, as the exposed users; and (iii) send a notification to each of the exposed users indicating they were in the predetermined contagious proximity to the symptomatic user.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### DESCRIPTION OF VARIOUS EMBODIMENTS

Numerous embodiments are described in this application, and are presented for illustrative purposes only. The described embodiments are not intended to be limiting in any sense. The invention is widely applicable to numerous embodiments, as is readily apparent from the disclosure herein. Those skilled in the art will recognize that the present invention may be practiced with modification and alteration without departing from the teachings disclosed herein. Although particular features of the present invention may be described with reference to one or more particular embodiments or figures, it should be understood that such features are not limited to usage in the one or more particular embodiments or figures with reference to which they are described.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

As used herein and in the claims, two or more parts are said to be “coupled”, “connected”, “attached”, “joined”, “affixed”, or “fastened” where the parts are joined or operate together either directly or indirectly (i.e., through one or more intermediate parts), so long as a link occurs. As used herein and in the claims, two or more parts are said to be “directly coupled”, “directly connected”, “directly attached”, “directly joined”, “directly affixed”, or “directly fastened” where the parts are connected in physical contact with each other. As used herein, two or more parts are said to be “rigidly coupled”, “rigidly connected”, “rigidly attached”, “rigidly joined”, “rigidly affixed”, or “rigidly fastened” where the parts are coupled so as to move as one while maintaining a constant orientation relative to each other. None of the terms “coupled”, “connected”, “attached”, “joined”, “affixed”, and “fastened” distinguish the manner in which two or more parts are joined together.

Further, although method steps may be described (in the disclosure and/or in the claims) in a sequential order, such methods may be configured to work in alternate orders. In other words, any sequence or order of steps that may be described does not necessarily indicate a requirement that the steps be performed in that order. The steps of methods described herein may be performed in any order that is practical. Further, some steps may be performed simultaneously.

As used herein and in the claims, a first element is said to be ‘communicatively coupled to’ or ‘communicatively connected to’ or ‘connected in communication with’ a second element where the first element is configured to send or receive electronic signals (e.g. data) to or from the second element, and the second element is configured to receive or send the electronic signals from or to the first element. The communication may be wired (e.g. the first and second elements are connected by one or more data cables), or wireless (e.g. at least one of the first and second elements has a wireless transmitter, and at least the other of the first and second elements has a wireless receiver). The electronic signals may be analog or digital. The communication may be one-way or two-way. In some cases, the communication may conform to one or more standard protocols (e.g. SPI, I²C, Bluetooth®, or IEEE™ 802.11).

As used herein and in the claims, a group of elements are said to ‘collectively’ perform an act where that act is performed by any one of the elements in the group, or performed cooperatively by two or more (or all) elements in the group.

Some elements herein may be identified by a part number, which is composed of a base number followed by an alphabetical or subscript-numerical suffix (e.g. 112a, or 112₁). Multiple elements herein may be identified by part numbers that share a base number in common and that differ by their suffixes (e.g. 112₁, 112₂, and 112₃). All elements with a common base number may be referred to collectively or generically using the base number without a suffix (e.g. 112).

As used herein and in the claims, “up”, “down”, “above”, “below”, “upwardly”, “vertical”, “elevation” and similar terms are in reference to a directionality generally aligned with (e.g. parallel to) gravity. The terms “forward”, “forwardly” and similar terms are in reference to a directionality generally that is transverse (e.g. perpendicular) to gravity and directed away from workstation 100. Accordingly, the terms “rear”, “rearwardly” and similar terms are in reference to a directionality that is generally transverse (e.g. perpendicular) to gravity and directed towards workstation 100. However, none of the terms referred to in this paragraph imply any particular alignment between elements. For example, a first element may be said to be “vertically above” a second element, where the first element is at a higher elevation than the second element, and irrespective of whether the first element is vertically aligned with the second element.

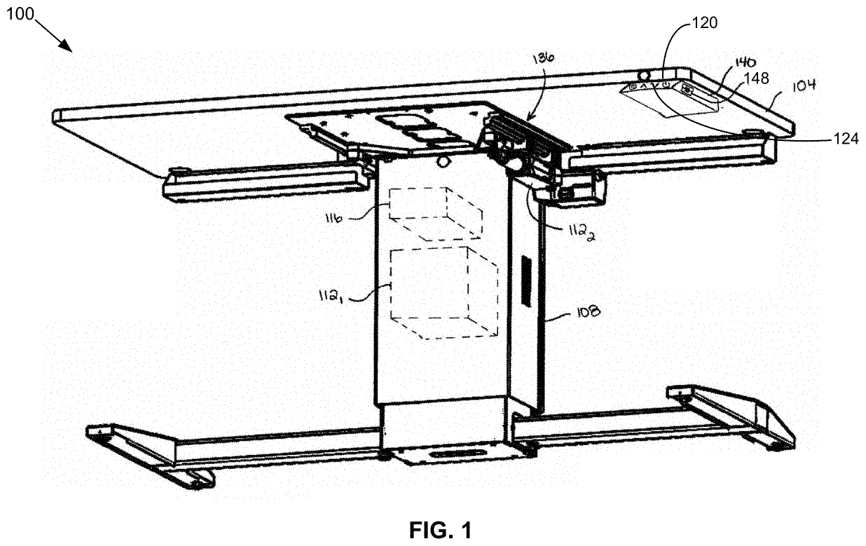

FIG. 1 depicts an example power-actuated workstation 100. As shown, workstation 100 includes a tabletop 104 and a vertical support 108. Tabletop 104 may provide a user work surface, such as to support documents, a computer, computer monitor(s), or other user articles. Vertical support 108 may support tabletop 104 above the ground at an elevation which is convenient for the user to interact with the user articles on tabletop 104.

As used herein and in the claims, the “ground” is a common surface that supports the workstation and any users at the workstation. The ground may be an indoor or outdoor floor covering (e.g. hardwood flooring, tiles, carpet, concrete, patio stones, or gravel), or a natural uncovered surface (e.g. grass, or soil).

Workstation 100 may include one or more actuators 112 that are operable to move tabletop 104 (e.g. linearly, rotationally, or arcuately) relative to the ground. In the example shown, workstation 100 includes a vertical actuator 112₁ that is operable to change the vertical position (i.e. elevation) of tabletop 104 above the ground, and a horizontal actuator 112₂ that is operable to change the horizontal position of tabletop 104 over the ground.

Activation of actuator(s) 112 may be controlled by a workstation controller 116. Workstation controller 116 may activate actuator(s) 112 (e.g. power actuator(s) 112 to execute a movement) in response to signals from user operable controls 120. For example, user operable controls 120 may include directional buttons 124 that a user can press to signal the workstation controller 116 to activate the actuator(s) 112 responsible for moving tabletop 104 in the selected direction (e.g. up, down, in, or out). That is, user operable controls 120 may send control signals to workstation controller 116, and in response, workstation controller 116 may activate actuator(s) 112 in accordance with the control signals. In the example shown, user operable controls 120 are provided on a control unit 140 that is mounted below tabletop 104. However, it will be appreciated that user operable controls 120 may be alternatively located in any user accessible location. For example, control unit 140 may be seated on tabletop 104 without being rigidly mounted. Alternatively, user operable controls 120 may be integrated within a component of workstation (e.g. tabletop 104 or vertical support 108).

An actuator 112 can be any device suitable to move tabletop 104 relative to the ground when activated by drive controller 116. An actuator 112 may include an electrically powered or electrically activated prime mover (i.e. source of motive power). For example, actuator 112 may include an electric motor (e.g. to drive a linear actuator, such as a leadscrew actuator), a solenoid (e.g. to provide linear motion directly, or to operate a valve), or a pump (e.g. to move fluid for activating a piston cylinder). Alternatively or in addition, an actuator 112 may be fluidly powered or fluidly activated. For example, actuator 112 may include a hydraulic or pneumatic device (e.g. a piston cylinder). Optionally, actuator 112 may include a mechanical transmission which may alter the directional characteristic of the prime mover (e.g. convert rotary to linear movement or vice versa), and/or provide mechanical advantage (e.g. multiply output force or torque). For example, actuator 112 may include one or more of gears, belts, screws, bar linkages, racks, or levers.

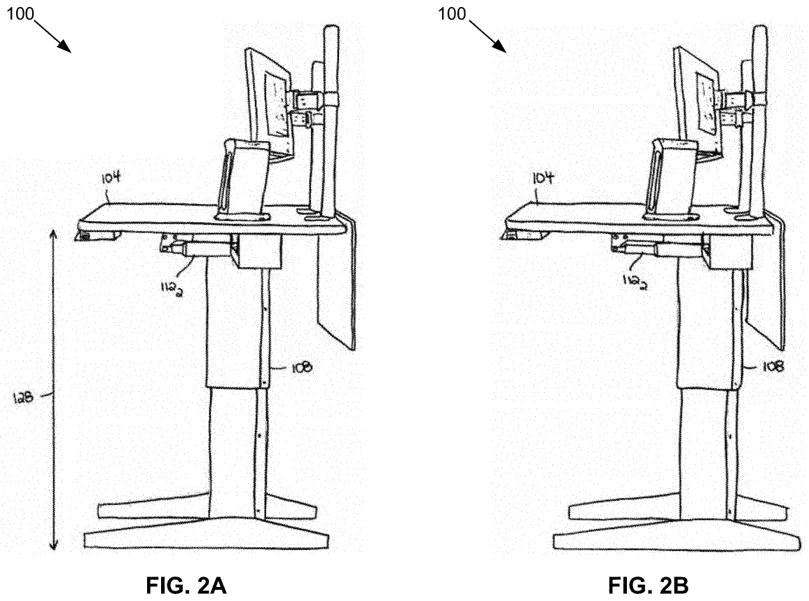

Reference is now made to FIGS. 2A-2D, which show tabletop 104 moved to different positions by actuators 112 (FIG. 1). In the example shown, vertical actuator 112₁ (FIG. 1) is part of vertical support 108 and acts to extend and retract vertical support 108. For example, vertical actuator 112₁ may be operable to extend/retract vertical support 108 between a raised height (FIGS. 2A-2B, e.g. standing height) and a lowered height (FIGS. 2C-2D, e.g. sitting height). Depending on the application of workstation 100 (e.g. as an office desk for computer and paperwork), tabletop 104 may have a standing tabletop height 128 of between 0.9 m and 1.5 m for use by a standing user, and a seated tabletop height 132 of between 0.5 m and 0.8 m for use by a seated user. In some cases, vertical actuator 112₁ (FIG. 1) can be stopped at many or every height between the raised and lowered heights. This allows workstation 100 to accommodate users of different body heights, and to accommodate different user postures (e.g. an upright posture when typing, or a leaning posture when drawing or handwriting).

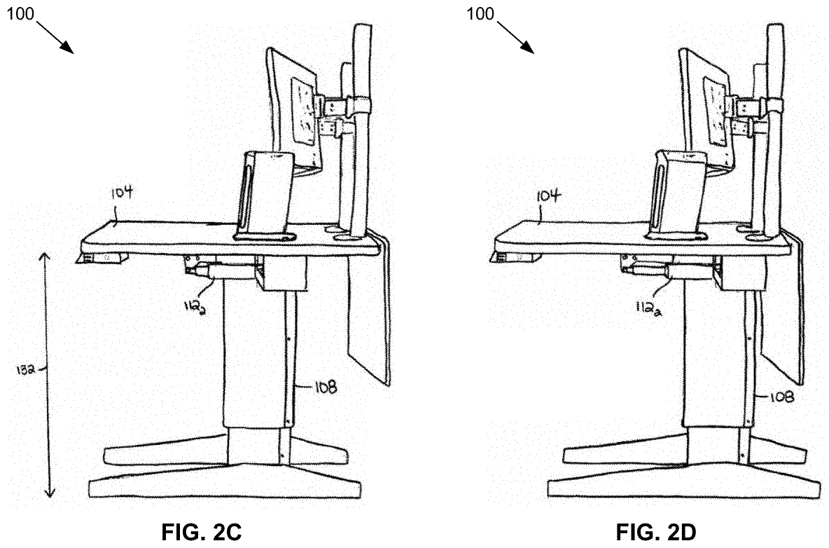

Horizontal actuator 112₂ may form part of the connection between tabletop 104 and vertical support 108. For example, horizontal actuator 112₂ may be operable to move tabletop 104 relative to the ground and vertical support 108 in a direction towards or away from a user position. As shown, this allows horizontal actuator 112₂ to move tabletop 104 between a rearward position (FIGS. 2A and 3A) and a forward position (FIGS. 2B and 3B). In the example shown, tabletop 104 is mounted to vertical support 108 by a rail assembly 136 (FIG. 1), and horizontal actuator 112₂ is a linear actuator (e.g. motorized leadscrew or rack and pinion) that moves tabletop 104 along the rail assembly 136 between the rearward position (FIGS. 2A and 2C) and the forward position (FIGS. 2B and 2D).

In some embodiments, there may be multiple actuators 112 that operate simultaneously or in succession to move tabletop 104 in one direction (e.g. along a linear, rotary, or curved path). For example, workstation 100 may include two or more spaced apart vertical supports 108 to provide greater stability in the case of a large tabletop 104, each vertical support 108 may include a vertical actuator 112₁ (FIG. 1), and all of the vertical actuators 112₁ (FIG. 1) may be activated simultaneously to move tabletop 104 between raised and lowered positions. As an example, FIG. 3 shows a power-actuated workstation 100 that includes two spaced apart vertical supports 108. As shown, each vertical support 108 has its own vertical actuator 112. In response to signals from user operable controls 120, drive controller 116 may activate both vertical actuators 112 simultaneously to move tabletop 104 between raised and lowered positions.

Various embodiments disclosed herein are directed at systems and methods for mitigating transmission of a contagious infection within a shared workspace. The shared workspace may include one or more floors in an office building or a classroom, for example. In particular, methods and systems disclosed herein relate to obtaining completion of a contagion control procedure in connection with a power-actuated workstation. At a high level, these methods and systems obtain completion of the contagion control procedure by inhibiting the tabletop of the workstation from moving away from an uncomfortable tabletop height in response to determining that the user has not completed the contagion control procedure.

In another aspect, methods and systems disclosed herein relate to contact tracing of a contagious infection in a shared workspace having a plurality of workstations. At a high level, these methods and systems notify users who may have been exposed to the contagious infection based on their proximity to workstations known to have been used by contaminated users during a predetermined preceding period.

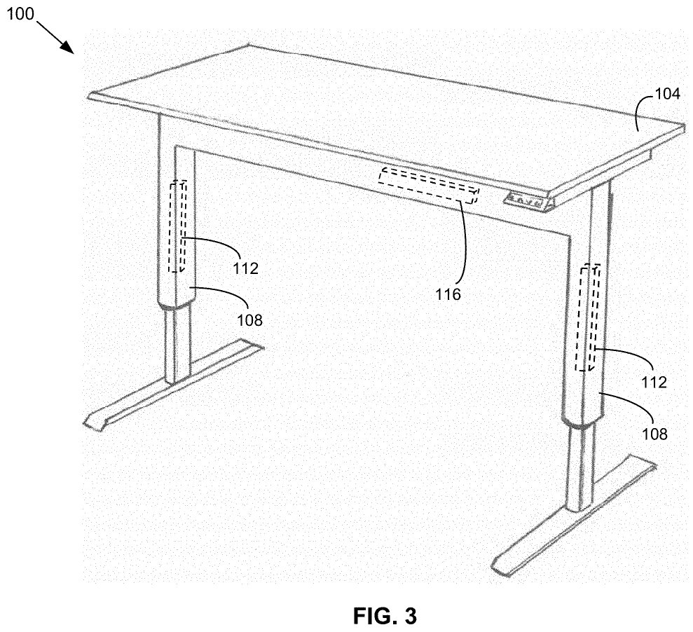

Reference is now made to FIG. 4, which shows a schematic illustration of a workplace safety system 200. As shown, workplace heath system 200 includes a server computer 204 communicatively coupled a workstation controller 116. For simplicity of illustration, only one workstation controller 116 is shown connected to server computer 204. However, multiple workstation controllers 116 may be concurrently connected to server computer 104. Each workstation controller 116 is associated with a corresponding workstation 100 (FIG. 3). In the example shown, workstation controller 116 is drivingly connection to first and second actuators 112. Workstation controller 116 and actuators 112 may be physically connected (e.g. by hardwiring) so that workstation controller 116 may power corresponding actuators 112 accordingly. In an alternative embodiment, a workstation controller 116 may be provided for each actuator 112 (e.g. one workstation controller 116 for a vertical actuator 112 and one workstation controller 116 for a horizontal actuator 112). In another alternative embodiment, a workstation controller 116 may control several actuators 112 (e.g. one workstation controller 116 to synchronize several vertical actuators 112, or one workstation controller 116 to control both vertical and horizontal actuators 112).

In the example shown, server computer 204 includes a processor 208, memory 212 and wireless module 216. Each of memory 212 and wireless module 216 are communicatively coupled to processor 208, directly or indirectly. In some embodiments, server computer 204 includes multiple of any one or more (or all) of processor 208, memory 212 and/or wireless module 216. In some embodiments, server computer 204 does not include one or more of memory 212 and wireless module 216. For example, server computer 204 may not include memory 212, and/or may not wireless module 216. In some embodiments, server computer 204 is a single, unitary device that houses all of its subcomponents (processor 208 and memory 212). In other embodiments, server computer 204 is composed of two or more discrete devices that are communicatively coupled to each other, that collectively include all of the subcomponents of server computer 204 (processor 208 and memory 212), and that collectively provide the functionality described herein.

In the example shown, workstation controller 116 includes a processor 144, memory 148, user operable controls 120, wireless module 152, presence sensor 156, display 160, card reader 164, power input 168, and power output 172. In some embodiments, workstation controller 116 includes multiple of any one or more (or all) of processor 144, memory 148, user operable controls 120, wireless module 152, presence sensor 156, display 160, card reader 164, power input 168, and power output 172. In some embodiments, workstation controller 116 does not include one or more of user operable controls 120, wireless module 152, presence sensor 156, display 160, card reader 164, power input 168, and power output 172. For example, workstation controller 116 may not include user operable controls 120, and/or may not include wireless module 152, and/or may not include presence sensor 156, and/or may not include display 160, and/or may not include card reader 164, and/or may not include power input 168, and/or may not include power output 172.

Each of memory 148, user operable controls 120, wireless module 152 presence sensor 156, display 160, card reader 164, power input 168, and power output 172 may be communicatively coupled to processor 144, directly or indirectly. In some embodiments, workstation controller 116 is a single, unitary device that houses all of its subcomponents (processor 144, memory 148, etc.). In other embodiments, workstation controller 116 is composed of two or more discrete devices that are communicatively coupled to each other, that collectively include all of the subcomponents of workstation controller 116 (processor 144, memory 148, etc.), and that collectively provide the functionality described herein.

The schematic of FIG. 4 illustrates the connection of server computer 204 to workstation controller 116. Server computer 204 may be communicatively connected to one or more workstation controllers 116 though a physical (i.e. wired) connection and/or a wireless connection (e.g., over a wireless network). Wireless modules 152 and 216 of workstation controller 116 and server computer 204, respectively, can be radios that communicate utilizing the CDMA, GSM, GPRS, or Bluetooth protocol according to such standards as 802.11 a, 802.11 b, 802.11 g or 802.11n, for example. Accordingly, wireless modules 152 and 216 may be used to communicate and/or relay signals between workstation controller 116 and server computer 204.

Memory 148 and 212 of workstation controller 116 and server computer 204, respectively, can include volatile memory (e.g. random access memory (RAM)) or non-volatile storage (e.g. ROM, flash memory, hard disk drive, solid-state drive, or other types of non-volatile data storage). In some embodiments, memory 148, 212 stores one or more applications for execution by corresponding processor 144, 208. The applications correspond with software modules including computer executable instructions to perform processing for the functions and methods described below. In some embodiments, some or all of memory 148, 212 may be integrated with corresponding processor 144, 208. For example, processor 144 may be a microcontroller (e.g. Microchip™ AVR, Microchip™ PIC, or ARM™ microcontroller) with onboard volatile and/or non-volatile memory.

In the example shown, both workstation controller 116 and server computer 204 are connected to an optional remote storage device 220. These connections can allow both workstation controller 116 and server computer 204 to communicate and/or relay signals with remote storage device 220. Remote storage device 220 may provide additional storage in cases where memory 148 of workstation controller 116 and memory 212 of server computer 204 provide insufficient storage. As an example, remote storage device 220 may include cloud storage. In at least one embodiment, only one of workstation controller 116 and server computer are connected to remote storage device 220.

Generally, processors 144 and 208 can execute applications, computer readable instructions or programs. The applications, computer readable instructions or programs can be stored in memory 148 and/or memory 212, or can be received from remote storage devices 220 across a wireless access network (WAN) or another suitable IP network (e.g. local access network (LAN)/storage area (SAN)). When executed, the applications, computer readable instructions or programs can configure processor 144 (or multiple processors 144) of workstation controller 116 and processor 208 (or multiple processors 208) of server computer 204 to collectively perform the acts described herein.

As discussed above, user operable controls 120 are manually operable (i.e. operable by deliberate physical interaction with a user's body part, such as a finger) to send command signals to activate actuators 112. For example, user operable controls 120 may include one or more of tactile or capacitive buttons 124 (FIG. 4A-4B), switches, sliders, knobs, or touch screens. When activated by the user, user operable controls 120 may signal processor 144 and, in response, processor 144 may select one or more actuator movements, or a regimen of actuator movements, and operate actuator(s) 112 accordingly.

In some embodiments, workstation controller 116 includes one or more presence sensors 156. As used herein and in the claims, a presence sensor 156 may be any device that can sense an indication (i.e. a presence reading) that a user is present at workstation 100 (FIG. 1), and communicate that presence reading (by wire or wirelessly) to processor 144. Presence sensor 156 may include, for example one or more (or all) of a pressure pad, a thermal sensor (e.g. infrared camera), a beam break sensor, and an ultrasonic sensor.

Display 160 can be any suitable display that provides visual information. For example, display 160 can be a flat-screen monitor for presenting visual information, such as tabletop height, user messages, time, etc.

In some embodiments, workstation controller 116 includes one or more card readers 164. As used herein and in the claims, a card reader 164 may be any device that can read a user's ID card (e.g., by scanning, swiping, or tapping the ID card on the card reader 164), and communicate user information associated with the ID card (by wire or wirelessly) to processor 144.

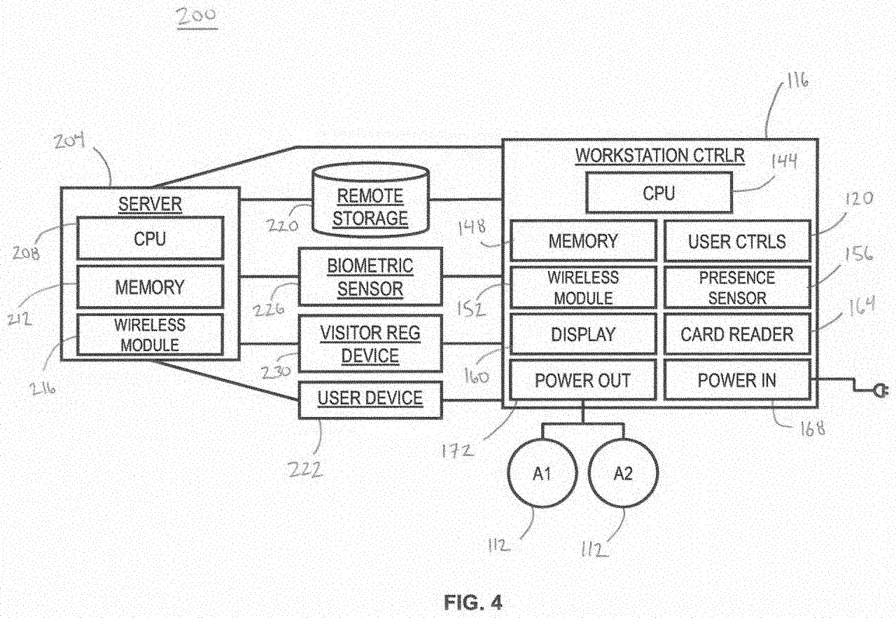

Reference is now made to FIG. 5, which depicts a flowchart illustrating an example process 500 of obtaining completion of a contagion control procedure. Processor 144 (or multiple processors 144) of workstation controller 116 and processor 208 (or multiple processors 208) of server computer 204 can be configured to collectively execute process 500.

Step 502 includes transmitting control signals to actuator(s) 112 to move tabletop 104 (FIG. 3) to an uncomfortable tabletop height. At the uncomfortable tabletop height, tabletop 104 may be at an elevation that provides a user with poor ergonomics for interacting with user articles on tabletop 104 (e.g. keyboard, mouse, monitor, etc.). In some embodiments, the uncomfortable tabletop height is between 85 cm and 105 cm above the ground. In other embodiments, the uncomfortable tabletop height is between 90 cm and 100 cm above the ground.

The uncomfortable tabletop height positions tabletop 104 at an elevation that a user may find unsuitable for standing or seated use. That is, the uncomfortable tabletop height may be too high for comfortable seated use and too low for comfortable standing use. With tabletop 104 at such an elevation, a user can be deterred from using workstation 100 without first moving tabletop 104 to an elevation conducive to comfortable seated use or comfortable standing use. In at least one embodiment, the uncomfortable tabletop height is within 30-70% of a transition between standing tabletop height 128 (FIGS. 2A-2B) and seated tabletop height 132 (FIGS. 2C-2D) (i.e. the uncomfortable tabletop height may be equal to the seated tabletop height 132 plus 0.3 to 0.7 times the difference between the standing tabletop height 128 and the seated tabletop height 132). In some embodiments, the uncomfortable tabletop height may be between 0.7 m and 1.1 m, such as for example, 0.85 m to 1.05 m, such as approximately 0.8 m to 0.9 m. In some embodiments, the uncomfortable tabletop height may be at or near (e.g. within 5 cm of) the lowest settable height for the workstation, and therefore too low to comfortably use for most users. For example, the uncomfortable height may be less than 0.5 m. In some embodiments, the uncomfortable tabletop height may be at or near (e.g. within 5 cm of) the highest settable height for the workstation, and therefore be too high to comfortably use for most users. For example, the uncomfortable tabletop height may be greater than 1.5 m.

In some embodiments, the uncomfortable height may be between (e.g. within 30-70% of a transition between) the seated and standing heights of the last person recorded using the workstation. In some embodiments, the uncomfortable height may be lower than (e.g. by 5 cm or more) the seated height of the last person recorded using the workstation. In some embodiments, the uncomfortable height may be higher than (e.g. by 5 cm or more) the standing height of the last person recorded using the workstation.

In some embodiments, the uncomfortable tabletop height may be between (e.g. within 30-70% of a transition between) the average seated and average standing heights of the previous persons recorded using the workstation. In some embodiments, the uncomfortable tabletop height may be lower than (e.g. by 5 cm or more) the average or lowest seated height of the previous persons recorded using the workstation. In some embodiments, the uncomfortable tabletop height may be higher than (e.g. by 5 cm or more) the average or highest standing height of the previous persons recorded using the workstation. The “previous persons” recorded using the workstation may be the previous predetermined number of recorded persons. The predetermined number may be 2 or more, such as 3-10 people. In some embodiments, the “previous persons” recorded using the workstation during a predetermined period. The predetermined period may be a number of hours (e.g. 24 or more hours), days (e.g. 1 or more days), business days (e.g. 1 or more business days), weeks (e.g. 1 or more weeks), or months (e.g. 1 or more months).

Step 504 includes detecting presence of a user at workstation 100. Presence of a user at workstation 100 can be detected in multiple ways. In one embodiment, detecting presence of a user at workstation 100 can include receiving a communication from a user device 222 (FIG. 4). The schematic of FIG. 4 illustrates the connection of a user device 222 to each of server computer 204 and workstation controller 116. User device 222 may be a smartphone, laptop, tablet, ID card, or set-top box, for example. User device 222 can be connected to each of server computer 204 and workstation controller 116 in any way that allows server computer 204 and workstation controller 116 to communicate and/or relay signals with user device 222. As an example, server computer 204 may be connected to a user device 222 across a wireless access network (WAN). As another example, workstation controller 116 may be connected to a user device 222 using Bluetooth. Alternatively, or in addition, each of server computer 204 and workstation controller 116 may be connected to a user device 222 through a wired connection (e.g., USB). In some examples (e.g. where the user device 222 is an ID card), user device 222 may be passive (i.e. having no power source) and reflect signals sent to it by workstation 100 (e.g. as in RFID).

In at least one embodiment, a user may be able to raise and lower tabletop 104 of workstation 100 (FIG. 1) via workstation controller 116, for example, by accessing a website or running a program on user device 222 (e.g., a smartphone or laptop). That is, user device 222 may send control signals to workstation controller 116, and in response, workstation controller 116 may activate actuator(s) 112 in accordance with those control signals.

Referring again to FIG. 5, the communication received at step 504 may be a signal received as part of establishing a Bluetooth paring between a user device 222 and workstation controller 116, for example. As another example, the communication received may be in response to a user “signing on” or otherwise connecting to workstation controller 116 with a user device 222. As noted above, a user device 222 may be an ID card associated with the user. In these cases, the communication received at step 504 may be in response to a user scanning their ID card at card reader 164 (FIG. 4).

A user profile associated with the communication received at step 504 may be accessed. For example, the communication at step 504 may include user credentials (e.g. username and password, or similar) associated with the user profile. Alternatively, or in addition, the communication may include a device ID (e.g., MAC ID or UDID) associated with the user profile. In addition to general information, such as the user's name and contact information, each user profile may store preferences associated with its associated user. For example, this may include preferred standing and seated tabletop heights stored in association with workstation 100. User profiles may be stored in one or more of memory 148 of workstation controller, memory 212 of server computer 204, remote storage device 220, and user device 222.

In at least one embodiment, transmitting control signals at step 502 may be performed after detecting presence of the user at step 504. In these embodiments, the control signals transmitted at step 502 can instruct actuator(s) 112 to move tabletop 104 to an uncomfortable tabletop height that is 30-70% of a transition between the preferred standing tabletop height and the preferred seated tabletop height for that particular user. Accordingly, the uncomfortable tabletop height may be user specific.

Step 506 includes determining whether the user has completed a contagion control procedure. The contagion control procedure may include any procedure designed to mitigate spread of a contagious virus in a shared workspace and that requires user participation to complete. As an example, the contagion control procedure may include a heath questionnaire formulated to assess whether the responding individual has symptoms associated with a contagious infection (e.g., Coronavirus, Flu, Streptococcus, etc.). Alternatively, the contagion control procedure may include receiving user biometric information (e.g., heart rate, blood pressure, respiratory rate, a measurement of oxygen saturation, glucose concentration, body temperature, etc.). The user biometric information may be received from one or more biometric sensors 226 (FIG. 4). Biometric sensors 226 may include, for example, wearable devices (e.g., smart watch, wrist bands, ear pieces, skin sensors, etc.) and standalone biometric devices that may be able to connect to workstation controller 116 and/or server computer 204 (e.g., using Bluetooth, Wi-Fi, etc.) and measure or determine various biometric data (e.g., health metrics) of its user.

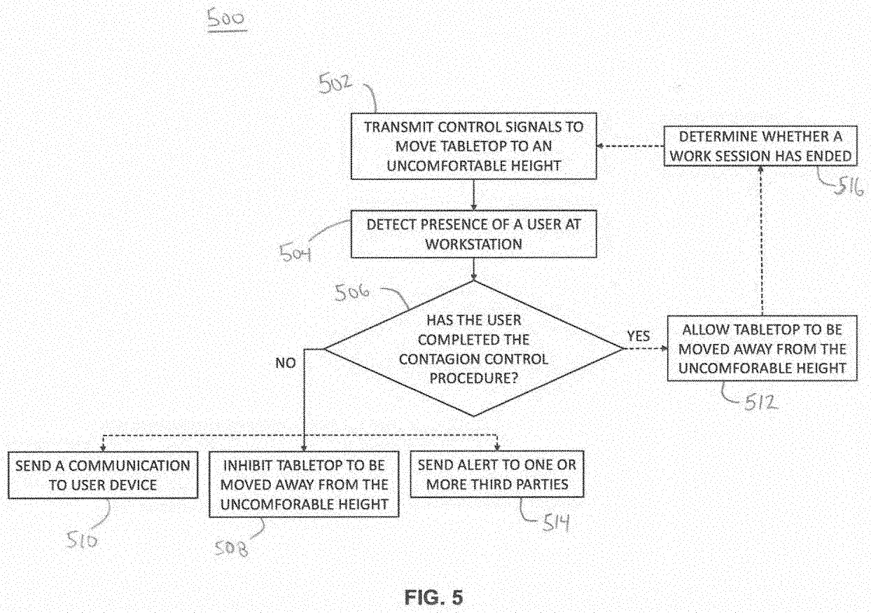

The contagion control procedure can also facility organizational compliance with government-mandated regulations and policy of the particular region. The contagion control procedure may be customized according to the specific needs of the shared workspace and/or to changes in government-mandated regulations or policy. FIGS. 6A to 6F depict a smartphone 222 displaying successive steps of an example contagion control procedure on its screen. The example contagion control procedure includes five steps, i) symptoms, ii) workstation cleaning, iii) hand washing, iv) physical distancing, and v) mask wearing, displayed successively on smartphone 222 from FIG. 6A to FIG. 6E. FIG. 6F depicts smartphone 222 displaying a confirmation that each element the contagion control procedure has been completed. In the illustrated example, the user may interact with the presented user-interface to complete each element of the contagion control procedure (e.g. complete the presented survey), and their responses may be stored in memory (e.g. in memory 212, remote storage 220, and/or memory 148) where it is (or will be when user device 222 connects to server 204 or workstation controller 116) accessible to server 204 and/or workstation controller 116 to assess whether the user has completed the contagion control procedure.

The determination of whether the user has completed the contagion control procedure can include determining the user profile indicates that the contagion control procedure was not completed. More particularly, such a determination can include determining the user profile indicates that a contagion control procedure was not completed within a predetermined preceding period. In some embodiments, predetermined preceding period is between 5 minutes and 24 hours. In other embodiments, predetermined preceding period is between 15 minutes and 12 hours. As an example, if the predetermined preceding period is 60 minutes and the user profile indicates that the contagion control procedure was completed 65 minutes ago, it will be determined that the user has not completed the contagion control procedure within the predetermined preceding period.

In response to determining that the user has not completed the contagion control procedure, step 508 includes inhibiting tabletop 104 to move away from the uncomfortable tabletop height in accordance with manual user instructions and/or automated user preferences (e.g. stored in association with user profiles). This can include non-responsiveness to control signals associated with manual user instructions and/or automated user preferences. For example, workstation controller 116 may not activate actuator(s) 112 according to control signals received from user operable controls 120 (FIG. 1) and/or user device 222 (FIG. 4).

Thus, the user may be unable to move tabletop 104 with user operable controls 120 without having completed the contagion control procedure in the predetermined preceding period. Alternatively, or in addition, workstation controller 116 may inhibit actuator(s) 112 from automating movement of tabletop 104 to one of the standing tabletop height and the seated tabletop height stored in association with the user profile of that user. Effectively, by inhibiting tabletop 104 from moving away from the uncomfortable tabletop height in accordance with manual user instructions and/or automated user preferences, the user is required to complete the contagion control procedure if they wish to move tabletop 104 to a comfortable tabletop height. In addition to physical discomfort, the user may experience peer pressure in the shared workspace from users who observe that the user's workstation tabletop 104 is at a clearly uncomfortable height.

In some embodiments, in response to determining that the user has not completed the contagion control procedure, step 510 includes sending a communication to the user device prompting the user to complete the contagion control procedure. As an example, the communication may be an email with a link to the contagion control procedure. In cases where user device 222 operates a software application or web-based application that is associated with workstation controller 116 and/or server computer 204, the communication may be viewable via the application.

Conversely, in response to determining that the user has completed the contagion control procedure, step 512 includes allowing tabletop 104 to move away from the uncomfortable tabletop height in accordance with manual user instructions and/or automated user preferences. In this case, workstation controller 116 may activate actuator(s) 112 according to control signals received from user operable controls 120 and/or user device 222. Alternatively, or in addition, workstation controller 116 may instruct actuator(s) 112 to automate movement of tabletop 104 to one of the standing tabletop height and the seated tabletop height stored in association with the user profile of that user. Thus, if it is determined that the user has completed the contagious control procedure at step 508, workstation controller 116 may be responsive to control signals associated with manual user instructions and/or automated user preferences.

In an alternative embodiment, step 504 can include detecting presence of a user at workstation 100 by one or more presence sensors 156 (FIG. 4). As discussed above, a presence sensor 156 may be any device that can sense an indication (i.e. a presence reading) that a user is present at workstation 100, and communicate that presence reading to processor 144. In these embodiments, determining that the user has not completed a contagion control procedure at step 506 includes receiving no communication from a user device 222 for a predetermined check-in duration from the detection at step 504. In some embodiments, the predetermined check-in duration is between 30 seconds and 5 minutes. Such a situation may occur when a user's presence is detected at workstation 100 by presence sensor(s) 156, but the user does not attempt to connect (i.e. “sign-in”) to workstation controller 116 and/or server computer 204 with a user device 222 (e.g. ID card, smartphone, etc.). In these cases, the user may be using workstation 100 with tabletop 104 at an uncomfortable tabletop height and/or using workstation 100 while not in compliance with the contagion control procedure.

In response to determining that the user has not completed the contagion control procedure, step 514 can include sending an alert to one or more third parties indicating a workstation ID and/or a workstation location associated with workstation 100. Server 204 and/or workstation 116 may store (e.g. in memory 212, 220, and/or 148) or have access to third party communication details (e.g. email address, phone number, instant messaging username, etc.) to which the alert is sent. As an example, the third parties may include an organization's health and safety officer, a security guard, and/or a facility manager. In this way, a third party can go to the workstation 100 identified in the alert to request that the user complete the contagion control procedure and/or leave the shared workspace (i.e. if they refuse). In some embodiments, the third party may include users who are nearby (e.g. within a predetermined distance of) the workstation 100 identified in the alert (e.g. they are logged on to nearby workstations).

In at least one embodiment, step 516 includes determining whether a work session has ended. Determining that the work session has ended may include receiving no communication has from user device 222 for a predetermined timeout duration. This may be the result of user device 222 disconnecting from workstation controller 116 and/or server computer 204, thereby indicating that the user intends to leave workstation 100 (or has already left the vicinity of workstation 100). In some embodiments, the predetermined timeout duration is between 1 and 300 seconds. In response to determining that the work session has ended, step 502 may be performed (or repeated). That is, control signals may be transmitted to actuator(s) 112 that instruct actuator(s) 112 to move to tabletop 104 to the uncomfortable tabletop height.

Alternatively, or in addition, determining that the work session has ended at step 516 may be based on presence readings from presence sensor(s) 156. Returning to FIG. 4, memory 148, 212 may store a termination protocol. In response to determining that the presence readings from the presence sensor 156 indicate a work session has ended at workstation 100 (FIG. 1), processor 144 may generate and send signals to operate the associated workstation actuator(s) 112 according to the termination protocol stored in memory. The termination protocol may include one or more actuator movements including, for example instructing actuator(s) 112 to move tabletop to the uncomfortable tabletop height between the seated tabletop height and the standing tabletop height (i.e. perform step 502).

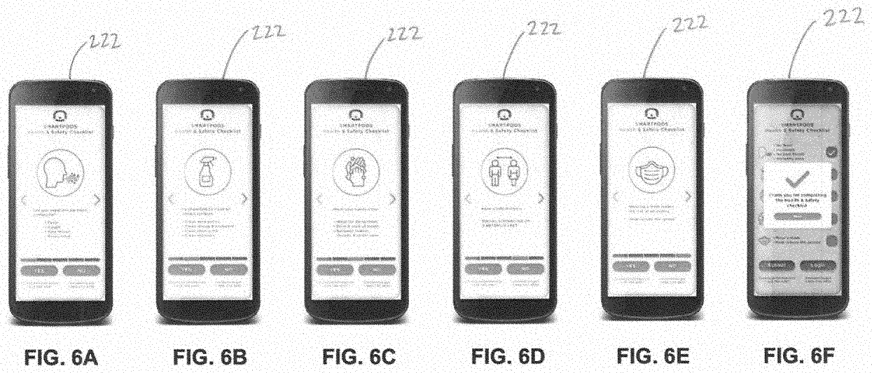

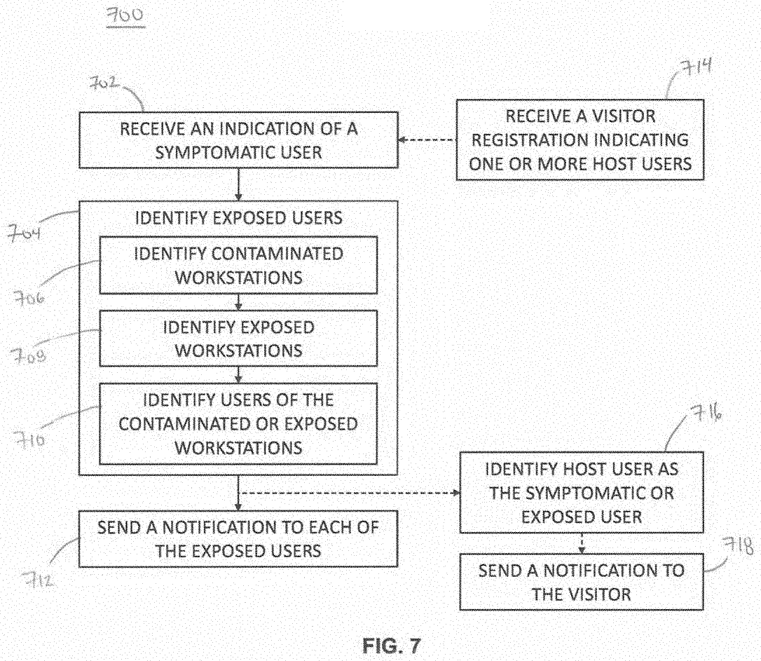

Reference is now made to FIG. 7, which depicts a flowchart illustrating an example method 700 of contact tracing in a shared workspace having a plurality of workstations. Server computer 104 and the workstation controllers 116 of the plurality of workstations 100 can be configured to collectively perform method 700. The shared workspace may include one or more floors in an office building or a classroom, for example. Each of the plurality of workstations in the shared workspace has a workstation controller 116 communicatively coupled to the server computer 204 (FIG. 4). The plurality of workstations 100 may be any one of the power-actuated workstations depicted in FIGS. 1 to 3, or a workstation without the capability of tabletop height adjustment. As an example, FIG. 8 shows a workstation 100 that includes wheels 176 connected at the bottom of vertical support 108 to facilitate mobility. As shown, workstation 100 has a workstation controller 116 seated atop a tabletop 104, but does not include actuator(s) operable to move tabletop 104.

Referring again to FIG. 7, step 702 includes receiving an indication that a symptomatic user of a plurality of users has one or more symptoms associated with a contagious infection. The indication may be based on or more of wearable health monitor data, user biometric information, and health questionnaire responses, for example.

The schematic of FIG. 4 illustrates the connection of a biometric sensor 226 to each of server computer 204 and workstation controller 116. For simplicity of illustration, only one biometric sensor 226 is shown connected to each of server computer 204 and workstation controller 116. However, multiple biometric sensors 226 may be concurrently connected to server computer 104 and/or workstation controller 116. In an alternative embodiment, one or more biometric sensors 226 may be connected to only one of server computer 204 and workstation controller 116.

Biometric sensors 226 may include, for example, wearable devices (e.g., smart watch, wrist bands, ear pieces, skin sensors, etc.) and standalone biometric devices that may be able to connect to workstation controller 116 and/or server computer 204 (e.g., using Bluetooth, Wi-Fi, etc.) and measure or determine various biometric data (e.g., health metrics) of its user. Health metrics of the user may include, but are not limited to: heart rate; blood pressure; respiratory rate; a measurement of oxygen saturation; glucose concentration; balance and posture; acceleration; a measurement of perspiration; body temperature; weight; caloric expenditure; and brain activity. The workstation controller 116 may receive measurements for health metrics at any time, periodically (e.g., every five minutes) and/or continuously. The health metrics may be analyzed (e.g. compared to baselines) to determine whether the user may have a contagious infection.

Users of the shared workspace may be periodically required to submit responses to health questionnaires. Individual questions on the health questionnaires may be formulated to assess whether the user may have symptoms associated with one or more types of contagious infection. In this way, a user's response to the health questionnaires may be analyzed to determine whether that user may have a contagious infection.

Step 704 includes identifying exposed users of the plurality of users that were in a predetermined contagious proximity to the symptomatic user during a predetermined preceding period. This identification can include intermediary steps 706, 708 and 710 discussed in turn below.

Step 706 includes identifying one or more contaminated workstations of the plurality of workstations where the symptomatic user was identified as present during the predetermined preceding period. In some embodiments, the predetermined preceding period is between 1 day and 30 days. In other embodiments, the predetermined preceding period is between 5 days and 14 days. The predetermined preceding period may be selected according to the incubation period associated with a particular contagious infection (e.g. Coronavirus, Flu, Meningitis, etc.). For example, if the preceding period is 14 days, any workstation 100 in the shared workspace where the symptomatic user was a recorded occupant during the preceding 14 days may be identified as a contaminated workstation.

Step 708 includes identifying each other workstation of the plurality of workstations, which are located in the predetermined contagious proximity to the one or more contaminated workstations, as exposed workstations. In some embodiments, the predetermined contagious proximity is between 1 m and 50 m. In other embodiments, the predetermined contagious proximity is between 2 m and 10 m. For example, if the predetermined contagious proximity is 10 m, any workstation 100 in the shared workspace that is located within a 10 m radius of each contaminated workstation may be identified as an exposed workstation. The predetermined contagious proximity may be based upon the transmissibility of the relevant contagious infection (e.g. Coronavirus, Flu, Meningitis, etc.), such as whether the contagious infection is spread by physical contact, fluid exchange, and/or is airborne. Memory 212 of server computer 204 and/or remote storage device 220 may store a location for each workstation of the plurality of workstations in the shared workspace so that all workstations within a predetermined contagious proximity of a contaminated workstation can be identified. Alternatively, or in addition, memory 212 and/or remote storage device 220 may store relative distances between each workstation in the shared workplace.

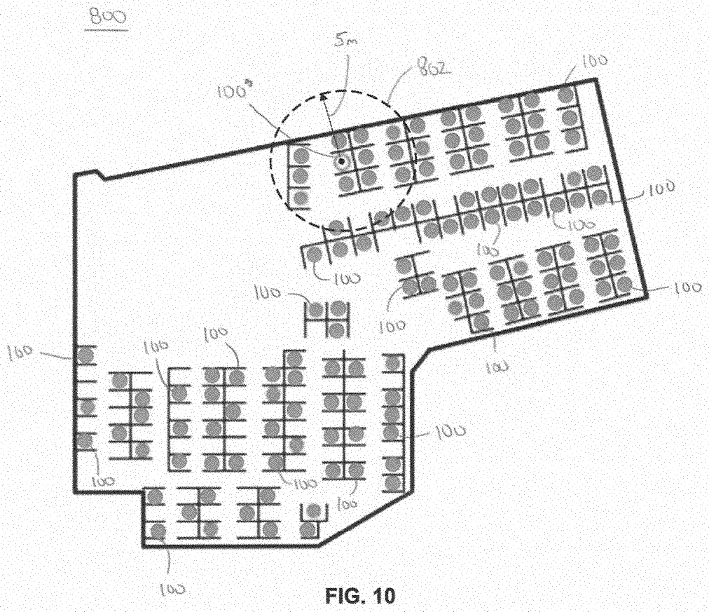

FIG. 10 depicts a schematic illustration of a floor plan 800 showing the locations of workstations 100. As example, if workstation 100′ has been identified as a contaminated workstation (at step 706) and the predetermined contagious proximity is 5 m, any other workstations 100 falling entirely and/or partially and/or having a center point within contagious proximity circle 802 may be identified as an exposed workstation.

Alternatively, in some embodiments, the predetermined contagious proximity may include any workstation that neighbors the contaminated workstation(s). In these embodiments, any workstation 100 in the shared workspace that neighbors (e.g. neighboring office or cubicle) a contaminated workstation may be identified as an exposed workstation.

Referring again to FIG. 7, step 710 includes identifying all other users of the plurality of users, who were identified as present at any of the exposed workstations or the contaminated workstations during the predetermined preceding period, as exposed users. Continuing with the example above, any user who was a recorded occupant at the exposed workstation(s) or contaminated workstations(s) during the preceding 14 days may be identified as exposed users.

Memory 212 of server computer 204, memory 148 of workstation controller 116 and/or remote storage device 220 may store occupancy periods for each of the plurality of users at each of the plurality of workstations. Occupancy periods may be stored in real time, or a regular interval (every 5 minutes), for example. In effect, occupancy periods provide records of which user was at which workstation at which time. The occupancy periods are retrievable from memory at step 706 to determine where the symptomatic user was identified as present during the predetermined preceding period. Similarly, occupancy periods are retrievable from memory at step 710 to determine exposed users who were identified as present at any of the exposed workstations or the contaminated workstations during the predetermined preceding period.

Step 712 includes sending a notification to each of the exposed users indicating they were in the predetermined contagious proximity to the symptomatic user. Server 204 and/or workstation 116 may store (e.g. in memory 212, 220, and/or 148) or have access to user communication details (e.g. email address, phone number, instant messaging username, etc.) to which the notification is sent. For example, the user communication details may be included in user profiles. The notification may identify remedial actions for the exposed employees to perform. Remedial actions, for example, may include advising the exposed user to stay home from work, to self-isolate for a prescribed period (e.g. the incubation period of the infection or the contagious period of the infection, such as for example 14 days), or to consult a doctor for testing.

The schematic of FIG. 4 illustrates the connection of a visitor registration device 230 to each of server computer 204 and workstation controller 116. For simplicity of illustration, only one visitor registration device 230 is shown connected to each of server computer 204 and workstation controller 116. However, multiple visitor registration devices 230 may be concurrently connected to server computer 104 and/or workstation controller 116. Visitor registration devices 230 can be connected to each of server computer 204 and workstation controller 116 in any way that allows server computer 204 and workstation controller 116 to communicate and/or relay signals with visitor registration device 230 (e.g. using Wi-Fi or Bluetooth). In an alternative embodiment, one or more visitor registration device 230 may be connected to only one of server computer 204 and workstation controller 116.

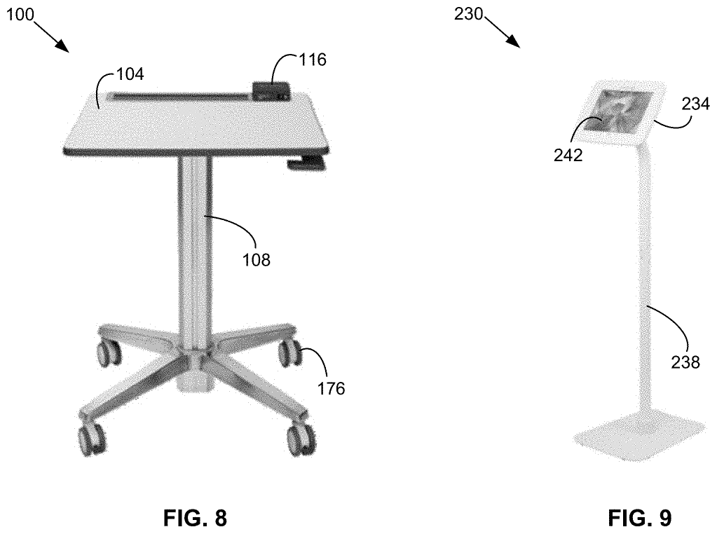

FIG. 9 depicts an example visitor registration device 230. As shown, visitor registration device may include a computing device (e.g. tablet) 234 supported above the ground by a vertical stand 238. Computing device 234 may operate a software application or web-based application that is associated with workstation controllers 116 and/or server computer 204. In the example shown, the application is accessible via a user interface 242 (e.g. touchscreen). One or more visitor registration devices 230 may be located in a reception area of the shared workspace to provide easy access by visitors.

Step 714 includes receiving, from the one or more visitor registration devices 230, a visitor registration for a visitor to the shared workspace. The visitor registration can indicate one or more host users of the plurality of users. That is, with user interface 242, the visitor can select the individuals with which they are meeting. Memory 212 of server computer 204, memory 148 of workstation controller 116 and/or remote storage device 220 may store the submitted visitor registrations.

Step 716 includes identifying one of the one or more host users as:
(i) the symptomatic user during the predetermined preceding period, or
(ii) one of the exposed users.

Step 718 includes sending a notification to the visitor indicating they were in the predetermined contagious proximity to the symptomatic user. The notification can identify remedial actions for the exposed employees to perform. Remedial actions, for example, may include advising the visitor to self-isolate for a prescribed period (e.g. the incubation period of the infection or the contagious period of the infection, such as for example 14 days), or to consult a doctor for testing.

In at least one embodiment, visitor registration requires the visitor to complete a contagion control procedure at the visitor registration device 230 using user interface 242. The contagion control procedure may be similar to the one shown in FIGS. 6A to 6F. The visitor may be denied entry to the shared workspace if they refuse to complete the contagion control procedure. As discussed above, a contagion control procedure may include a heath questionnaire formulated to assess whether the responding individual has symptoms associated with a contagious infection (e.g., Coronavirus, Flu, Streptococcus, etc.). In these cases, the visitor may also be denied entry to the shared workspace if their responses indicate that they may have a contagious infection.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

Items
Item 1: A method of obtaining completion of a contagion control procedure, the method being performed in connection with a workstation having a tabletop and one or more actuators operable to move the tabletop, the method comprising:
   detecting presence of a user at the workstation, the user having a seated tabletop height and a standing tabletop height stored in association with the workstation;
   transmitting, to the one or more actuators, control signals that instruct the one or more actuators to move the tabletop to an uncomfortable tabletop height between the seated tabletop height and the standing tabletop height; and
   in response to determining that the user has not completed the contagion control procedure, inhibiting the tabletop from moving away from the uncomfortable tabletop height in accordance with at least one of manual user instructions and automated user preferences.
Item 2: The method of any other item, wherein said transmitting is performed before said detecting.
Item 3: The method of any other item, wherein said detecting comprises:
   receiving a communication from a user device; and
   accessing the user profile associated with the communication.
Item 4: The method of any other item, wherein said determining comprises:
   determining the user profile indicates that the contagion control procedure was not completed.
Item 5: The method of any other item, wherein said determining comprises:
   determining the user profile indicates that the contagion control procedure was not completed within a predetermined preceding period.
Item 6: The method of any other item, wherein the predetermined preceding period is between 15 minutes and 12 hours.
Item 7: The method of any other item, wherein, in response to said determining the user profile indicates that the contagion control procedure was not completed within the predetermined preceding period, sending a communication to the user device prompting the user to complete the contagion control procedure.
Item 8: The method of any other item, wherein, in response to said determining that the user has not completed the contagion control procedure, sending a communication to the user device prompting the user to complete the contagion control procedure.
Item 9: The method of any other item, wherein the uncomfortable tabletop height is between 85 cm and 105 cm above the ground.
Item 10: The method of any other item, wherein, at the uncomfortable tabletop height, the tabletop is within 30-70% of a transition between the seated tabletop height and the standing tabletop height.
Item 11: The method of any other item, wherein said inhibiting comprises:
   inhibiting activation of the one or more actuators according to control signals associated with the at least one of the manual user instructions and the automated user preferences
Item 12: The method of any other item, further comprising:
   in response to determining that a work session has ended, transmitting, to the one or more actuators, control signals that instruct the one or more actuators to move to the tabletop to the uncomfortable tabletop height.
Item 13: The method of any other item, wherein said determining that the work session has ended comprises:
   receiving no communication from the user device for a predetermined timeout duration.
Item 14: A method of obtaining completion of a contagion control procedure, the method being performed in connection with a workstation having one or more actuators operable to move a tabletop of the workstation, the method comprising:
   transmitting, to the one or more actuators, control signals that instruct the one or more actuators to move the tabletop to an uncomfortable tabletop height between 85 cm and 105 cm above the ground;
   detecting presence of a user at the workstation; and
   in response to determining that the user has not completed the contagion control procedure, inhibiting the tabletop from moving away from the uncomfortable tabletop height in accordance with at least one of manual user instructions and automated user preferences.
Item 15: The method of any other item, wherein said transmitting is performed before said detecting.
Item 16: The method of any other item, wherein said detecting comprises:
   detecting presence of the user at the workstation by one or more presence sensors.
Item 17: The method of any other item, wherein said determining comprises:
   identifying that no communication has been received from a user device for a predetermined check-in duration from said detecting.
Item 18: The method of any other item, further comprising:
   in response to said determining, sending an alert to one or more third parties indicating at least one of a workstation ID and a workstation location associated with the workstation.
Item 19: A system for obtaining completion of a contagion control procedure in connection with a workstation having a tabletop and one or more actuators operable to move of the tabletop, the system comprising:
   a workstation controller configured to send control signals to the one or more actuators to move the tabletop;
   a server computer communicatively coupled to the workstation,
   each of the workstation controller and the server computer having one or more processors, the processors of the workstation controller and server computer configured to collectively:
      detect presence of a user at the workstation, the user having a seated tabletop height and a standing tabletop height stored in association with the workstation;
      transmit, to the one or more actuators, control signals that instruct the one or more actuators to move the tabletop to an uncomfortable tabletop height between the seated tabletop height and the standing tabletop height; and
      in response to determining that the user has not completed the contagion control procedure, inhibit the tabletop from moving away from the uncomfortable tabletop height in accordance with at least one of manual user instructions and automated user preferences.
Item 20: The system of any other item, wherein the processors of the workstation controller and server computer are configured to collectively:
   transmit the control signals that instruct the one or more actuators to move the tabletop to the uncomfortable tabletop height before detecting presence of the user at the workstation.
Item 21: The system of any other item, wherein to detect presence of the user at the workstation, the processors of the workstation controller and server computer are configured to collectively:
   receive a communication from a user device; and
   access a user profile associated with the communication.
Item 22: The system of any other item, wherein to determine that the user has not completed the contagion control procedure, the processors of the workstation controller and server computer are configured to collectively:
   determine the user profile indicates that the contagion control procedure was not completed.
Item 23: The system of any other item, wherein to determine that the user has not completed the contagion control procedure, the processors of the workstation controller and server computer are configured to collectively:
   determine the user profile indicates that the contagion control procedure was not completed within a predetermined preceding period.
Item 24: The system of any other item, wherein the predetermined preceding period is between 15 minutes and 12 hours.
Item 25: The system of any other item, wherein the processors of the workstation controller and server computer are configured to collectively:
   in response to determining the user profile indicates that the contagion control procedure was not completed within the predetermined preceding period, send a communication to the user device prompting the user to complete the contagion control procedure.
Item 26: The system of any other item, wherein the processors of the workstation controller and server computer are configured to collectively:
   in response to determining that the user has not completed the contagion control procedure, send a communication to the user device prompting the user to complete the contagion control procedure.
Item 27: The system of any other item, wherein the uncomfortable tabletop height is between 85 cm and 105 cm above the ground.
Item 28: The system of any other item, wherein the uncomfortable tabletop height is within 30-70% of a transition between the seated tabletop height and the standing tabletop height.
Item 29: The system of any other item, wherein, to inhibit the tabletop from moving away from the uncomfortable tabletop height, the processors of the workstation controller and server computer are configured to collectively:
   inhibit activation of the one or more actuators according to control signals associated with the at least one of the manual user instructions and the automated user preferences.
Item 30: The system of any other item, wherein, the processors of the workstation controller and server computer are configured to collectively:
   in response to determining that a work session has ended, transmit, to the one or more actuators, control signals that instruct the one or more actuators to move to the tabletop to the uncomfortable tabletop height.
Item 31: The system of any other item, wherein, to determine that the work session has ended, the processors of the workstation controller and server computer are configured to collectively:
   identify that no communication has been received from the user device for a predetermined timeout duration.
Item 32: A computer-readable medium comprising instructions executable by one or more processors, wherein the instructions when executed configure the one or more processors to collectively:
   detect presence of a user at a workstation, the user having a seated tabletop height and a standing tabletop height stored in association with the workstation;
   transmit, to one or more actuators of the workstation, control signals that instruct the one or more actuators to move a tabletop of the workstation to an uncomfortable tabletop height between the seated tabletop height and the standing tabletop height;
   in response to determining that the user has not completed a contagion control procedure, inhibit the tabletop from moving away from the uncomfortable tabletop height in accordance with at least one of manual user instructions and automated user preferences.
Item 33: A system for contact tracing in a shared workspace having a plurality of workstations, the system comprising:
a server computer having one or more processors;
   each of the plurality of workstations having a workstation controller communicatively coupled to the server computer, each workstation controller having one or more processors;
   the server computer and the workstation controllers of the plurality of workstations configured to collectively:
      receive an indication that a symptomatic user of a plurality of users has one or more symptoms associated with a contagious infection;
      identify exposed users of the plurality of users that were in a predetermined contagious proximity to the symptomatic user during a predetermined preceding period by
         identifying one or more contaminated workstations of the plurality of workstations where the symptomatic user was identified as present during the predetermined preceding period;
         identifying each other workstation of the plurality of workstations, which are located in the predetermined contagious proximity to the one or more contaminated workstations, as exposed workstations; and
         identifying all other users of the plurality of users, who were identified as present at any of the exposed workstations or the contaminated workstations during the predetermined preceding period, as the exposed users; and
      send a notification to each of the exposed users indicating they were in the predetermined contagious proximity to the symptomatic user.
Item 34: The system of any other item, further comprising:
   one or more data storage devices communicatively coupled to (a) the server computer, (b) one or more of the plurality of workstations, or both (a) and (b);
   the server computer and the workstation controllers of the plurality of workstations configured to collectively:
      store, in the one or more of the data storage devices, occupancy periods for each of the plurality of users at each of the plurality of workstations.
Item 35: The system of any other item, wherein the server computer and the workstation controllers of the plurality of workstations are configured to collectively:
   store, in the one or more of the data storage devices, a location for each workstation of the plurality of workstations in the shared workspace.
Item 36: The system of any other item, wherein the notification identifies remedial actions for the exposed employees to perform.
Item 37: The system of any other item, wherein the predetermined preceding period is between 5 days and 15 days.
Item 38: The system of any other item, wherein the predetermined contagious proximity is between 2 m and 10 m.
Item 39: The system of any other item, further comprising:
   one or more visitor registration devices communicatively coupled to communicatively coupled to (a) the server computer, (b) one or more of the plurality of workstations, or both (a) and (b);
   the server computer and the workstation controllers of the plurality of workstations configured to collectively:
      receive, from the one or more visitor registration devices, a visitor registration for a visitor to the shared workspace, the visitor registration indicating one or more host users of the plurality of users; and
      in response to identifying one of the one or more host users as (i) the symptomatic user during the predetermined preceding period or (ii) one of the exposed users, send a notification to the visitor indicating they were in the predetermined contagious proximity to the symptomatic user.
Item 40: The system of any other item, wherein the server computer and the workstation controllers of the plurality of workstations are configured to collectively:
   store, in the one or more of the data storage devices, the visitor registration.
Item 41: The system of any other item, wherein the one or more visitor registration devices are located in a reception area of the shared workspace.
Item 42: The system of any other item, wherein the server computer and the workstation controllers of the plurality of workstations are configured to collectively:
   transmit, to the one or more visitor registration devices, a communication prompting the visitor to complete a contagion control procedure.
Item 43: The system of any other item, wherein the indication is based on at least one of wearable health monitor data, user biometric information, and health questionnaire responses.
Item 44: A method of contact tracing in a shared workspace having a plurality of workstations, the method comprising:
   receiving an indication that a symptomatic user of a plurality of users has one or more symptoms associated with a contagious infection;
   identifying exposed users of the plurality of users that were in a predetermined contagious proximity to the symptomatic user during a predetermined preceding period by
      identifying one or more contaminated workstations of the plurality of workstations where the symptomatic user was identified as present during the predetermined preceding period;
      identifying each other workstation of the plurality of workstations, which are located in the predetermined contagious proximity to the one or more contaminated workstations, as exposed workstations; and
      identifying all other users of the plurality of users, who were identified as present at any of the exposed workstations or the contaminated workstations during the predetermined preceding period, as the exposed users; and
   sending a notification to each of the exposed users indicating they were in the predetermined contagious proximity to the symptomatic user.
Item 45: The method of any other item, further comprising:
   storing occupancy periods for each of the plurality of users at each of the plurality of workstations.
Item 46: The method of any other item, further comprising:
   storing a location for each workstation of the plurality of workstations in the shared workspace.
Item 47: The method of any other item, wherein the notification identifies remedial actions for the exposed employees to perform.
Item 48: The method of any other item, wherein the predetermined preceding period is between 5 days and 15 days.
Item 49: The method of any other item, wherein the predetermined contagious proximity is between 2 m and 10 m.
Item 50: The method of any other item, further comprising:
   receiving a visitor registration for a visitor to the shared workspace, the visitor registration indicating one or more host users of the plurality of users; and
   in response to identifying one of the one or more host users as (i) the symptomatic user during the predetermined preceding period or (ii) one of the exposed users,
   sending a notification to the visitor indicating they were in the predetermined contagious proximity to the symptomatic user.
Item 51: The method of any other item, further comprising:
   storing the visitor registration.
Item 52: The method of any other item, wherein the indication is based on at least one of wearable health monitor data, user biometric information, and health questionnaire responses.
Item 53: A computer-readable medium comprising instructions executable by one or more processors, wherein the instructions when executed configure the one or more processors to collectively:
   receive an indication that a symptomatic user of a plurality of users has one or more symptoms associated with a contagious infection;
   identify exposed users of the plurality of users that were in a predetermined contagious proximity to the symptomatic user during a predetermined preceding period by
      identifying one or more contaminated workstations of a plurality of workstations where the symptomatic user was identified as present during the predetermined preceding period;
      identifying each other workstation of the plurality of workstations, which are located in the predetermined contagious proximity to the one or more contaminated workstations, as exposed workstations; and
      identifying all other users of the plurality of users, who were identified as present at any of the exposed workstations or the contaminated workstations during the predetermined preceding period, as the exposed users; and
   send a notification to each of the exposed users indicating they were in the predetermined contagious proximity to the symptomatic user.

1. A system for contact tracing in a shared workspace having a plurality of workstations, the system comprising:
a server computer having one or more processors;
each of the plurality of workstations having a workstation controller communicatively coupled to the server computer, each workstation controller having one or more processors;
the server computer and the workstation controllers of the plurality of workstations configured to collectively:
receive an indication that a symptomatic user of a plurality of users has one or more symptoms associated with a contagious infection;
identify exposed users of the plurality of users that were in a predetermined contagious proximity to the symptomatic user during a predetermined preceding period by
identifying one or more contaminated workstations of the plurality of workstations where the symptomatic user was identified as present during the predetermined preceding period;
identifying each other workstation of the plurality of workstations, which are located in the predetermined contagious proximity to the one or more contaminated workstations, as exposed workstations; and
identifying all other users of the plurality of users, who were identified as present at any of the exposed workstations or the contaminated workstations during the predetermined preceding period, as the exposed users; and
send a notification to each of the exposed users indicating they were in the predetermined contagious proximity to the symptomatic user.

2. The system of claim 1, further comprising:
one or more data storage devices communicatively coupled to (a) the server computer, (b) one or more of the plurality of workstations, or both (a) and (b);
the server computer and the workstation controllers of the plurality of workstations configured to collectively:
store, in the one or more of the data storage devices, occupancy periods for each of the plurality of users at each of the plurality of workstations.

3. The system of claim 2, wherein the server computer and the workstation controllers of the plurality of workstations are configured to collectively:
store, in the one or more of the data storage devices, a location for each workstation of the plurality of workstations in the shared workspace.

4. The system of claim 1, wherein the notification identifies remedial actions for the exposed employees to perform.

5. The system of claim 1, wherein the predetermined preceding period is between 5 days and 15 days.

6. The system of claim 1, wherein the predetermined contagious proximity is between 2 m and 10 m.

7. The system of claim 1, further comprising:
one or more visitor registration devices communicatively coupled to communicatively coupled to (a) the server computer, (b) one or more of the plurality of workstations, or both (a) and (b);
the server computer and the workstation controllers of the plurality of workstations configured to collectively:
receive, from the one or more visitor registration devices, a visitor registration for a visitor to the shared workspace, the visitor registration indicating one or more host users of the plurality of users; and
in response to identifying one of the one or more host users as (i) the symptomatic user during the predetermined preceding period or (ii) one of the exposed users, send a notification to the visitor indicating they were in the predetermined contagious proximity to the symptomatic user.

8. The system of claim 7, wherein the server computer and the workstation controllers of the plurality of workstations are configured to collectively:
store, in the one or more of the data storage devices, the visitor registration.

9. The system of claim 7, wherein the one or more visitor registration devices are located in a reception area of the shared workspace.

10. The system of claim 7, wherein the server computer and the workstation controllers of the plurality of workstations are configured to collectively:
transmit, to the one or more visitor registration devices, a communication prompting the visitor to complete a contagion control procedure.

11. The system of claim 1, wherein the indication is based on at least one of wearable health monitor data, user biometric information, and health questionnaire responses.

12. A method of contact tracing in a shared workspace having a plurality of workstations, the method comprising:
receiving an indication that a symptomatic user of a plurality of users has one or more symptoms associated with a contagious infection;
identifying exposed users of the plurality of users that were in a predetermined contagious proximity to the symptomatic user during a predetermined preceding period by
identifying one or more contaminated workstations of the plurality of workstations where the symptomatic user was identified as present during the predetermined preceding period;
identifying each other workstation of the plurality of workstations, which are located in the predetermined contagious proximity to the one or more contaminated workstations, as exposed workstations; and
identifying all other users of the plurality of users, who were identified as present at any of the exposed workstations or the contaminated workstations during the predetermined preceding period, as the exposed users; and
sending a notification to each of the exposed users indicating they were in the predetermined contagious proximity to the symptomatic user.

13. The method of claim 12, further comprising:
storing occupancy periods for each of the plurality of users at each of the plurality of workstations.

14. The method of claim 12, further comprising:
storing a location for each workstation of the plurality of workstations in the shared workspace.

15. The method of claim 12, wherein the notification identifies remedial actions for the exposed employees to perform.

16. The method of claim 12, wherein the predetermined preceding period is between 5 days and 15 days.

17. The method of claim 12, wherein the predetermined contagious proximity is between 2 m and 10 m.

18. The method of claim 12, further comprising:
receiving a visitor registration for a visitor to the shared workspace, the visitor registration indicating one or more host users of the plurality of users; and
in response to identifying one of the one or more host users as (i) the symptomatic user during the predetermined preceding period or (ii) one of the exposed users, sending a notification to the visitor indicating they were in the predetermined contagious proximity to the symptomatic user.

19. The method of claim 12, wherein the indication is based on at least one of wearable health monitor data, user biometric information, and health questionnaire responses.

20. A computer-readable medium comprising instructions executable by one or more processors, wherein the instructions when executed configure the one or more processors to collectively:
receive an indication that a symptomatic user of a plurality of users has one or more symptoms associated with a contagious infection;
identify exposed users of the plurality of users that were in a predetermined contagious proximity to the symptomatic user during a predetermined preceding period by
identifying one or more contaminated workstations of a plurality of workstations where the symptomatic user was identified as present during the predetermined preceding period;
identifying each other workstation of the plurality of workstations, which are located in the predetermined contagious proximity to the one or more contaminated workstations, as exposed workstations; and
identifying all other users of the plurality of users, who were identified as present at any of the exposed workstations or the contaminated workstations during the predetermined preceding period, as the exposed users; and
send a notification to each of the exposed users indicating they were in the predetermined contagious proximity to the symptomatic user.
